(19) United States
(12) Patent Application Publication
(10) Pub. No.: US 2013/0274583 A1
(43) Pub. Date: Oct. 17, 2013

(54) Electrodes Adapted for Transmitting or Measuring Voltages Through Hair

(71) Applicant: Sandy L. Heck, Los Angeles, CA (US)

(72) Inventor: Sandy L. Heck, Los Angeles, CA (US)

(21) Appl. No.: 13/860,642

(22) Filed: Apr. 11, 2013

Related U.S. Application Data
(60) Provisional application No. 61/622,562, filed on Apr. 11, 2012.

(51) Int. Cl. A61N 1/04; A61B 5/0492; A61B 5/04

(52) U.S. Cl. CPC A61N 1/0484; A61B 5/04001; A61B 5/0492
USPC 600/383; 607/139

(57) ABSTRACT

An electrode that functions alone, in clusters or arrays can be used to measure and/or deliver voltages through skin covered with hair by achieving reliable skin contact. In one form, a composite metal electrode has several raised points capable of passing through hair to contact the skin directly between hairs. Electrode points are distributed evenly over a flat circular or curved base to create a composite electrode or cluster in which all electrode points electrically couple to one another and to an output device. In another form of the invention, each electrode point is connected by a separate conductor wire to a selector that evaluates the signal during use and determines which of the electrode points are to be coupled to an output device.

### CROSS REFERENCE TO RELATED APPLICATIONS

I claim benefit of my provisional application 61/622,562, filed Apr. 11, 2012; provisional application 61/413661, filed Nov. 15, 2010, utility application Ser. No. 13/295446, filed Nov. 14, 2011, and utility application Ser. No. 13/632594, filed Dec. 1, 2012, which are hereby incorporated herein by reference.

### FIELD OF INVENTION

This invention generally relates to individual electrodes, to electrode clusters and to electrode arrays that can be used to effectively interface with skin through hair, preferably offering the ability to non-invasively apply electrical signals as well as to measure skin surface EMG voltages that arise in the body under skin areas covered with hair, such as the scalp without necessitating that the hair be shaved or parted.

### BACKGROUND AND POTENTIAL APPLICATIONS

Measuring voltages generated by living tissue is important in certain clinical and biomedical applications. Voltages generated by muscles (EMG or electromyography) are measured in a clinical setting to diagnose neuromuscular disorders. These voltages have also been successfully tested by the present applicant as command signals for prosthetics and other biomedical applications. Being able to non-invasively measure these signals with electrodes that rest on the skin surface is an important factor in their clinical use, since more invasive measurement modalities, including needle electrodes that penetrate the skin, screw electrodes, and implanted electrodes, introduce increased medical risks, increased discomfort, and ultimately decreased practical utility.

A major objective of this invention is to provide a new type of metal skin surface electrode that is capable of effectively measuring EMG, EKG and EEG voltages through hair. Traditional surface electrodes are not capable of measuring small voltages through thick hair, since the metal electrode must directly contact the skin, and the presence of hair disrupts the electrical connection between the skin and electrode surfaces, significantly degrading the signal and potentially introducing noise. When it is desired to measure surface voltages over skin areas with substantial hair, such as the scalp or chest, traditional electrodes require that the skin area be shaved or carefully parted beneath the electrode. Significant disadvantages are associated with this current method, since carefully parting the hair of the scalp requires the assistance of another individual, and shaving the scalp is aesthetically undesirable for most individuals. The present invention by contrast allows the electrode to be placed blindly over thick hair, without necessitating that the skin area be exposed with shaving or hair parting. The invention therefore allows voltages to be more easily measured over areas covered with hair as well as allowing such measurements on new individuals who would have been unwilling to shave for such testing. In addition to enabling scalp voltage measurements, the electrodes are ideally suited for measurement of surface voltages over any area covered in hair, including EKG through chest hair and the measurement of skin voltages in mammals with body fur.

In some clinical and biomedical application, voltages can also be applied to the skin through metal surface electrodes (electrical stimulation). The delivery of voltages through skin into muscles can serve diagnostic, rehabilitative, and prosthetic applications. The present invention describes a surface electrode that can also be used to effectively deliver voltages (electrical shocks) through skin that is covered with hair. Delivering voltages through thick hair with current electrodes can be problematic and dangerous, since the flow of current is disrupted by the presence of hair at the skin interface, and it risks injury to skin and surrounding tissue. For example, delivering a defibrillating shock during cardiac arrest can be complicated by thick chest hair, interfering with both diagnostic EKG measurements and the delivery of effective shocks.

In view of these and other deficiencies of the prior art, it is one object of the invention is to find a non-invasion way to provide good command signals from the mammalian body to an output device or as instructions to any electrical or electronic device such as a computer, e.g. to designate direction or a point on a screen or input to a memory device or as input to an actuator for performing a designated function, etc.

Another object is to make electrical contract through the hair more reliably without the hair being shaved or parted while at the same time being comfortable for the patient or other user.

Still another object is to make it possible for the patient to selectively adjust the pressure applied by the points of a multi-pointed electrode onto the skin.

Yet another is to make good electrical contact between the points of a multi-pointed electrode and the skin surface for conveying faint electrical currents generated by both nerve impulses and muscle contractions to a pickup device.

### SUMMARY OF THE INVENTION

The present invention provides an electrode that can function alone, in clusters or in arrays to effectively measure and/or deliver voltages through skin covered with hair by achieving reliable skin contact. In one form, a composite metal electrode has several raised points capable of passing through hair to contact the skin directly between hairs. In one form, electrode points are distributed evenly over a flat circular or curved base to create a composite electrode or cluster in which all electrode points are electrically coupled to one another and wherein the base is typically 6-10 mm in diameter with multiple points each rising about 3-5 mm from the base. In one embodiment, an electrode cluster is made of solid stainless steel or Ag/AgCl. Alternatively, it can be of any metal typically used in the construction of surface electrodes. In another form of the invention, each electrode point is connected by a separate conductor wire to a selector that evaluates the signal and determines which of the electrode points are to be coupled to an output device during use.

The foregoing features, objects and advantages of the invention will become apparent to those skilled in the art from the following detailed description of a preferred embodiment, especially when considered in conjunction with the accompanying drawings in which like numerals in the several views referred to corresponding parts.

### BRIEF DESCRIPTION OF THE DRAWINGS

FIG. 1 is a perspective view of a headset utilizing the invention.

FIG. 1A is a partial enlarged perspective view of a top portion of one of the electrode supports of FIG. 1 shown in position over a peri-auricular muscle with the skin removed.

FIG. 2 is a perspective view of one of the duplex electrodes on a larger scale to enable details to be more clearly seen.

FIG. 3 is a vertical cross-sectional view of the duplex electrodes shown in FIG. 2 with the points of the electrodes somewhat retracted.

FIG. 4 is a view similar to FIG. 3 showing the electrodes extended further toward a skin surface at the right to make a more firm electrical connection so as to reduce electrical resistance between the points of the electrode and the skin surface.

FIG. 5 is a perspective view showing an electrode headset in the form of an eyeglasses style framework with skin contacting the sensor electrodes thereon which are positioned adjacent the vestigial auricular nerves or muscles while being worn.

FIG. 6 is another view of the framework of FIG. 5 as it appears when placed on the head with the skin contacting sensor electrodes positioned to contact the skin over the vestigial auricular nerves or muscles.

FIG. 7 is an enlarged perspective view of a cluster of sensor electrodes as shown in FIGS. 1-4 shown to illustrate individual wires for each of the sensor electrodes at the back of the base supporting the electrode cluster.

FIG. 8 is a perspective view of another electrode headset in the form of an eyeglasses framework with a large array of skin contacting the sensor electrodes thereon of sufficient size to cover all of the peri-auricular nerves or muscles where electrical signals are likely to arise.

FIG. 9 is a diagrammatic side elevational view of the electrodes of FIG. 8 in which certain clusters of electrodes are programmed to be selected based upon the signals received from the body.

FIG. 10 is a view similar to FIG. 9 in which oval-shaped rings depict a selection of different groups of sensor electrodes based upon the signal quality received through those electrodes from the body.

### DETAILED DESCRIPTION OF THE PREFERRED EMBODIMENT

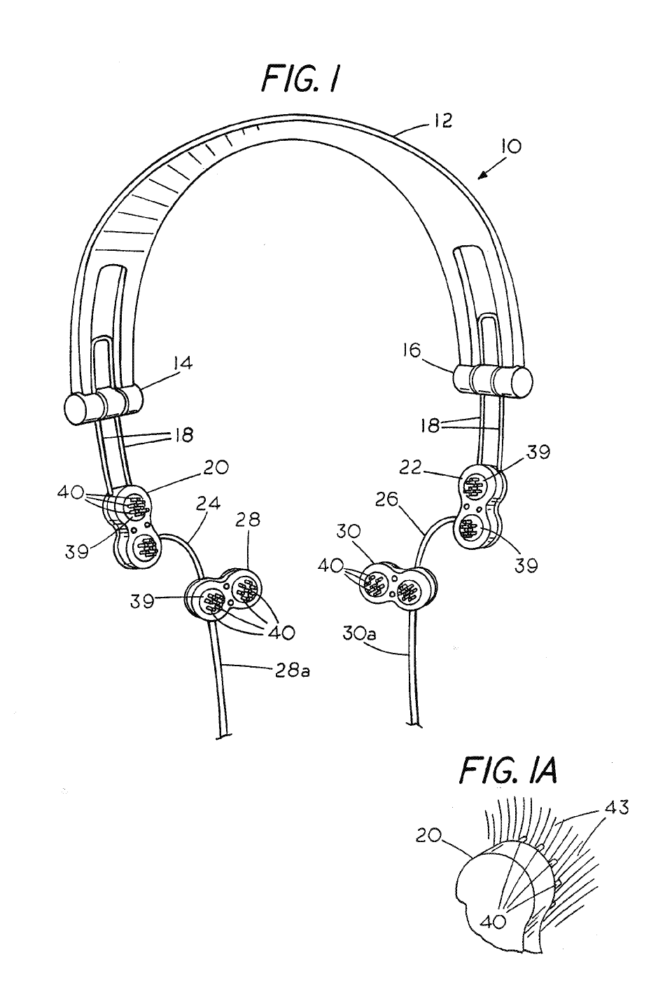

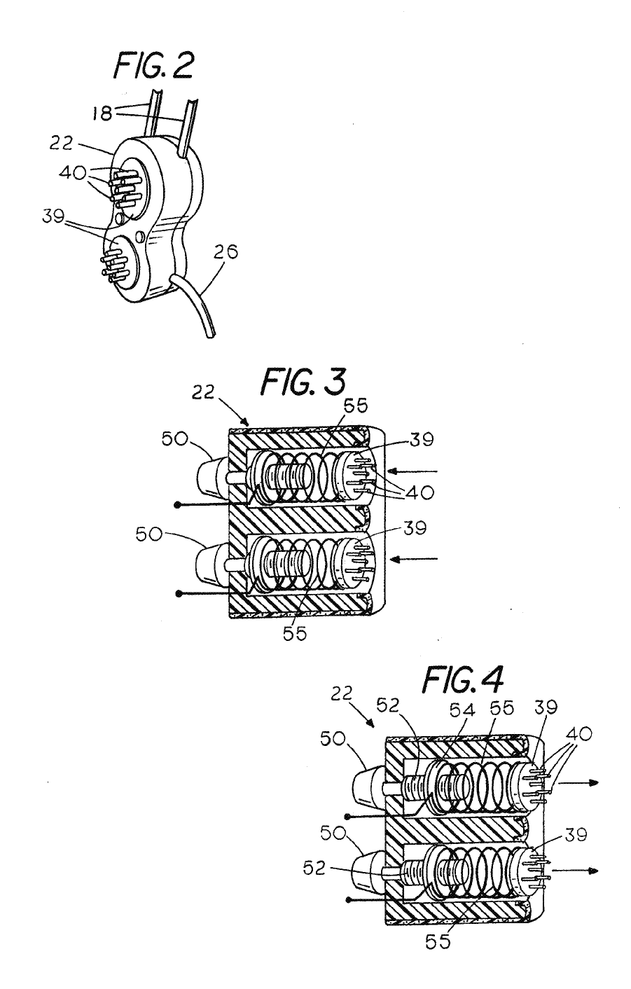

Referring now to FIGS. 1-4, the numeral 10 designates a headset having a resilient head band 12 to which are secured left and right brackets 14 and 16 having stiff wire electrode holders 18 extending from that are connected at their lower ends to duplex electrode assemblies 20 and 22 for the left and right side of the head respectively. Extending downwardly from the lower electrode set are support wires 24 and 26, which can be formed from stainless steel or other resilient material, to which laterally disposed duplex electrodes 28 and 30 are connected. Each duplex electrode 20, 22, 28, 30 has seven parallel, horizontally extending elongated generally cylindrical electrode points 40 formed from a good electrical conductor extending from a base plate 39. A cable 28a to carry a signal extends from electrodes 20 and 28 and a cable 30a extends from duplex electrode 30.

FIGS. 3 and 4 show how the user, by rotating anyone of the knobs 50, a pressure adjustment screw 52 which is threaded through a circular adjustment plate 54 can be used to slide the adjustment plate 54 toward the right to thereby extend the electrode points 40 further toward the skin of the patient so as to exert greater contact via helical spring 55 for pressing the points 40 more tightly into electrical contact with the skin to enhance the electrical connection by reducing electrical resistance at the skin surface. The spring 55 in this embodiment is positioned between the electrode and base plate 39, thereby applying a constant inward force on the electrode to further press the points 40 against the skin with an adjustable pressure. Individual electrode points 40 are shown in place over a peri-auricular muscle 43 (FIG. 1A) with the skin removed to enable the muscle to be seen. The electrodes are meant to be used “dry”, but the electrode could also be used with a conductive gel or cream. In one preferred embodiment, the side-walls of each electrode 40 have no slope. However, a slight draft angle of 2-3 degrees may be incorporated to improve manufacturability if desired. The diameter of a small electrode support or base 39 having a diameter 6-10 mm is used for optimally capturing EMG signals from small scalp muscles. As shown in FIGS. 1-4, the electrodes are paired so as to provide a duplex structure or differential electrode configuration with a distance typically of 3-6 mm between each base 39 to maximize the signal to noise ratio when measuring from small length muscles.

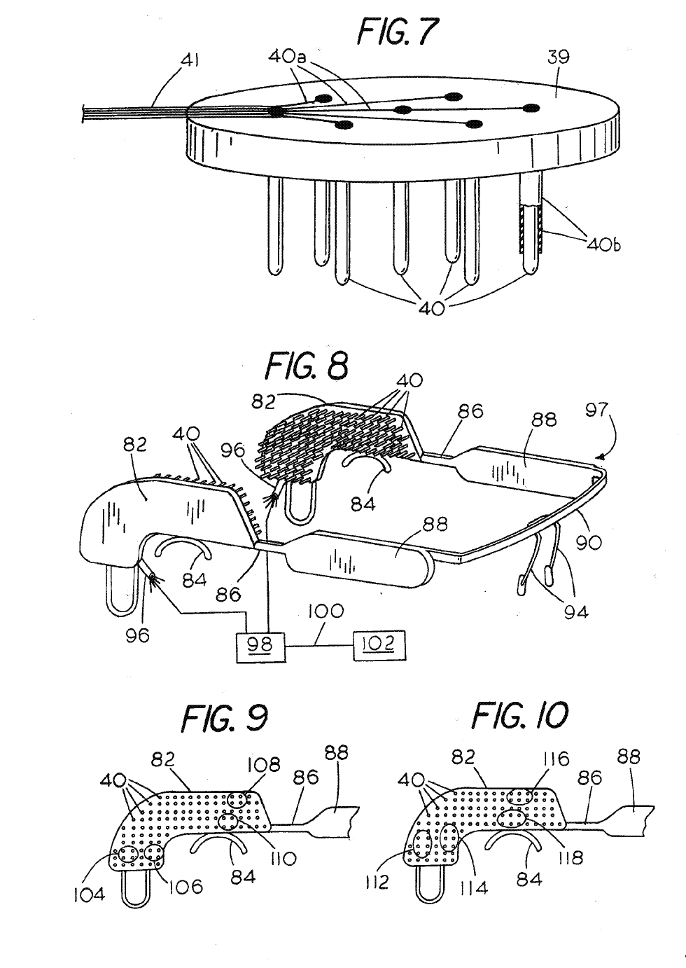

If desired, each electrode 40 can have an insulating covering, such as a tubular rubber coating 40b (FIG. 7), or a plastic cap (not shown), to insulate the electrode surfaces not contacting skin, while still leaving the tips of the pointed electrodes un-insulated. Alternatively, the electrode can be used without any insulating covering. In one preferred embodiment, the side-walls of each electrode 40 have no slope.

However, a slight draft angle of 2-3 degrees may be incorporated to improve manufacturability if desired. In one form of the invention, the electrodes are paired so as to provide a duplex structure in a differential electrode configuration (FIGS. 1- 4) with a distance of 3-6 mm between each base 39 is used to maximize the signal to noise ratio when measuring from small length muscles. A small electrode support or base 39 having a diameter 6-10 mm is used for optimally capturing EMG signals from small scalp muscles. Each electrode base 39 carrying several parallel electrode points is most preferably integrated into the headset and positioned bilaterally over scalp hair, to measure scalp muscles usually covered in hair, as shown in FIGS. 1, 5 and 6. In FIGS. 1-4 all projecting electrodes 40 in each cluster are connected to the same electrical wire that transmits electric current to or from the skin. However, each projecting electrode point 60 can be wired to a separate conductor wire as will be desired herein below.

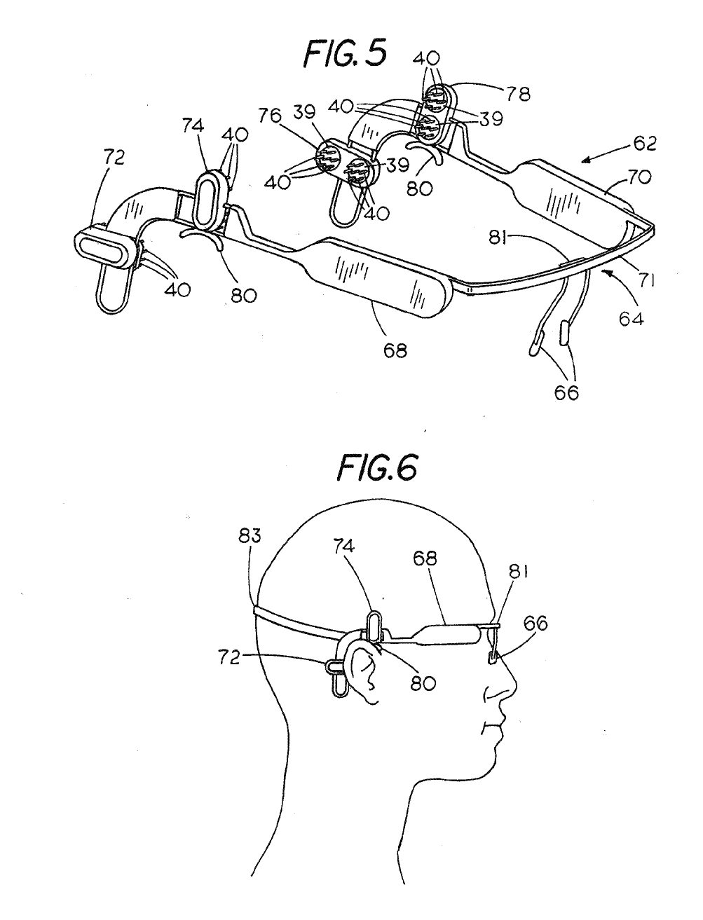

Refer now to FIGS. 5 and 6, which show a convenient way of positioning skin contacting sensors in close proximity to the vestigial peri-auricular nerves/muscles of the user. A support structure of a style similar to that used to mount lenses in eyeglasses is indicated generally by numeral 62 and has a plastic frame 64 with pads 66 for supporting the frame on the bridge of the wearer's nose. The pads 66 are preferably electrically conductive and function as a ground or reference electrode. It also has bows 68, 70 adapted to be supported by the ears of the wearer in the well-known manner. Ear rests are shown at 80. Disposed within the bows is the electronic signal processing circuitry as described in my co-pending applications referred to herein above. Mounted on the bows proximate their downward curve that wraps around the ears are one or more EMG electrode supports 72,74,76 and 78 of the type previously described so positioned that they will closely overlay the vestigial peri-auricular nerves and muscles, allowing pickup of EMG potentials when the support structure or frame 62 is being worn. The frame's bow members 68 and 70 also supports the electronics for performing signal processing and signal transmission function earlier described in my prior application Ser. No. 13/295446, filed Nov. 14, 2011. Printed wiring is preferably employed to connect the ground electrode 66 and the EMG electrode bases 72, 74, 76 and 78 to the electronic circuitry residing in the bows 68, 70.

The term “eyeglasses style headset” as used herein is intended to indicate any headset that extends along the side of the head on each side over the ears, anteriorly around the forehead, and which may be supported by the ears and optionally by a means of nose pads that contact the sides of the nose and head strap around the back of the head.

To enhance comfort for the user, ear pads, as at 80, are positioned to rest on the skin where the external ears join the head. A suitably shaped pad 81 may also be provided at the point where the front portion of the frame engages the forehead. As shown in FIG. 6, an elastic band or strap 83 may encircle the back of the wearer's head to more firmly compress the electrodes 72, 74, 76, 78 against the skin at the site of the vestigial nerves and muscles for more intimate contact and to more firmly secure the device 62 in place.

Refer now to FIG. 7, which illustrates how in the embodiments of FIGS. 5, 6 and 8-10 each of the individual sensor electrodes 40 has a separate lead wire 40a connected to it, each of which is individually insulated to provide a cable 41 made up of all of the individual lead wires 40a that together make up one of the signal cables 96 of FIG. 8. If desired, each electrode 40 can have an insulating covering, such as a tubular rubber coating 40b or a plastic cap (not shown), to insulate the electrode surfaces not contacting skin, while still leaving the tips of the pointed electrodes un-insulated. Alternatively, the electrode can be used without any insulating covering.

FIG. 8 illustrates another preferred form of electrode support structure 97 for sensor electrodes which holds an electrode array on each side of the head that is made up of a larger number, typically from about 20-80 sensor electrodes, each about 6-10 mm long and each directed toward the side of the head in alignment with the vestigial peri-auricular nerves and muscles 43 (FIG. 1A) on each side of the head. In this case, each of the sensor electrodes 40 in the array is supported upon a vertically disposed supporting plate 82 which as best seen in FIG. 8 extends rearwardly and curves downwardly at its rearward end behind the ear of the user which is located beneath a curved ear rest 84, which fits above the ear during use so that the electrode sensors 40 extend centrally into contact with the user's skin over the peri-auricular nerves and muscles on both sides of the head. Extending forwardly from each of the supports 82 is a bow 86 which has a forwardly-extending vertically-disposed section 88 at its forward end on each side of the head enclosing sensor circuitry as in FIGS. 5 and 6. The bows 86 are connected to one another by means of a forehead bar 90 on which a pair of electrically conductive nose pads 92 that establish an electrical ground to the nose are mounted by means of struts 94. The configuration of the framework is similar to that used for a pair of eyeglasses but without lenses. The framework provides a secure, comfortable and effective way of holding the sensor electrodes that comprise a wide array in contact with the skin on each side of the head over the entire area where the peri-auricular nerves and muscles are located for providing electrical contact capable of sensing EMG signals from the muscles under the control of the user for the purpose of performing any of a variety of selected function as described herein above and in my co-pending applications, Ser. No. 13/295446, dated Nov. 14, 2011 and Ser. No. 13/632592, dated Dec. 1, 2012, which are incorporated herein by reference. As described above in connection with FIG. 7, each of the electrode sensors 40 in FIG. 8 has its own separate electrical connection wire which is insulated and assembles with all of the similar wires into a single cord 96 with each of the individual lead wires being coupled to programmable control circuitry, which in turn is connected by a cable 100 to an indicator, a servo or other output device as described in any of the foregoing co-pending applications that is adapted to enable the user to provide visual or electronic signals for operating an output device or computer or to perform other desired functions as the user energizes the peri-auricular nerves in a particular way or pattern.

Refer now to FIGS. 9 and 10, wherein the same numerals refer to corresponding parts in FIG. 8. In this case, four circles have been drawn on the array of electrodes 40 to indicate groups of electrodes that are chosen by the selector 98, for example, a threshold-triggered operational amplifier to turn off all electrodes with signals below a certain selected threshold, so as to provide a resulting signal based upon the quality of the signals received by each electrode from the body tissue. For example, if the four electrodes in each of the circles at the lower right indicated at 104 and 106 are producing signals that are consistently over a predetermined threshold voltage, they will be chosen by the selector 98 using circuit components known to those skilled in the art for continued transmission to the output device or receiver 102 through the cable 100 while the other surrounding electrode sensors will be turned off and not used so that only the electrode sensors within the circles 104 and 106 are actually used. The circles 108 and 110 circumscribe other electrode sensors that are selected in the same manner as described above based upon their ability to produce a signal that is above a preselected threshold voltage. For example, if the invention is used by a patient with limited mobility, she could be given instructions to “signal a motion to the rear”. The selector 98 would then select an area circumscribing the electrode sensors such as indicated at 104 that are the most effective in signaling the desired rearward motion. Similarly, the electrodes within the circle 106 are selected as those that are most effective in signaling forward motion, those at 108 most effective in signaling upward motion and those circumscribed by the circle 110 as the most effective in signaling downward motion. Their criteria can be used for selecting different individual or groups of electrodes as those that are the most capable of producing any particular predetermined function while those electrodes outside the circled areas are turned off and not used while the others are in operation.

FIG. 10 shows how under different conditions certain other groups of electrode sensors 40 are identified and designated for use by the selector 98 which in this case are indicated by areas of a different shape such as oval areas at 112 to 118. In this way, the headset 97 which incorporates the relatively large electrode array that is shown mounted on the electrode support plate 82 includes certain electrodes chosen by the selector 98 based typically on the consistency of the signal and the signal-to-noise ratio or other quality that is above a predetermined threshold. A comparison between FIGS. 9 and 10 shows how different patterns of the selected electrode groups can change under various operating conditions. The selector 98 can identify a single electrode sensor point or a larger group of electrode points such as a cluster 116 which will then be grouped together electronically to represent one “electrode signal”. Variations in the size and arrangement of each electrode group will result from variations in the underlying sizes and locations of the EMG signals being measured when the user performs a selected function or indicate a particular direction or for performing other tasks or functions as the case may be. While various selector circuits can be employed for using only those signals above a certain threshold, one example of such a selector is a threshold actuated circuit, which is well-known per se to those skilled in the art that employs only signals above a selected signal-to-noise threshold. Other selector circuits of suitable known construction can also be used if desired. Signal conditioning circuitry of suitable known construction can also be used such as preamplifiers, noise clipper or limiter optionally with pulse shaping or digitizing. A signal converter is also helpful to rectify incoming signals to logic system levels. These circuit components per se are known to those skilled in the art.

It can thus be seen that providing each sensor electrode pin 40 with its own electrical wire 40a leading to the selector 98, it becomes possible to evaluate the quality of the signal produced by individual electrode pins 40 and thereby ignore signals generated by other electrode pins where the skin contact is poor or for any reason causing that location over the underlying muscle to be other than optimal so that the signal-to-noise characteristics are poor. Thus, while the electrode clusters described in FIGS. 1-8 are each the same size (each consisting, for example, of about seven pins), the embodiment of FIGS. 7-10 however provides a much larger number spread over an extended area of the skin encompassing a location where all of the signals from nerve impulses and muscle EMG signals are likely to occur. The length of each of the pin electrodes can be as long as needed and may be longer than previously disclosed, for example, about one centimeter in length since it has been found that certain individuals with long hair need electrode pins that can extend up to one centimeter or more. Thus, during operation, a large array of electrode points 40 throughout the entire support 82 will cover most or the entire skin surface overlying the target muscles. In this way, the large array shown in FIGS. 8-10 is able to eliminate the need for the pairs of electrode clusters as shown in the previous embodiments, such as the upper and lower pair indicated at 20 in FIG. 1. This is accomplished by utilizing only the individual electrode points that are the most effective are determined by the selector 98. In this case, the two regions such as the regions 104 and 106 of FIG. 9 are the best and most effective regions for capturing differential signals, such as by way of example, the directions “forward and reverse” with the cluster 104 indicating “reverse” and the cluster at 106 indicating “forward”.

A preferred mechanism for selecting each optimal electrode cluster such as 104 and 106 as the most effective in serving as the signal-producing electrode preferably utilizes signal-to-noise characteristic from each individual electrode point or projection. While the projections with poor signals are ignored by the selector 987, signals from the entire array are in this way modeled so that those from different clusters such as 104 and 106 serve as those that produce the clearest and most representative signal. In the example given, it is the EMG signals which best represent the intended contractions of peri-auricular muscles but different signals are selected when other muscles are used. During operation, the signal-to-noise threshold circuit incorporates components that cut out the signals that are not desired and allow the desired clusters of signals to be averaged which allow their combined signals to approximate that from a single electrode point 40.

In one preferred embodiment, selection process of selector 98 is software-driven and is rule-based, optionally incorporating genetic algorithms if desired to automatically select the best combination of signals from a large set of possibilities. The selector 98 thus identifies electrodes that produce the largest and most representative signal-to-noise ratio above a predetermined threshold and cuts off signals from the remaining electrodes. Generally, the higher the ratio, the better the signal. In one example, the electrode signals can be fed to an operational amplifier that compares the signal from each pin to a constant reference voltage and allows only those signals above a selected threshold to be transmitted on to whatever output device is being used.

A primary benefit of providing a large array of electrodes 40 covering the entire support 82 is that it allows the selection of electrode points that are the most effective to act as a single electrode source for adapting to variations in anatomy which exist between different people as well as enabling a single headset 96 to be dynamically modified electronically each time it is used by the same person used since the electrode points 40 will not always have the same signal characteristics, even for the same person each time they are used since one or more hairs may occasionally block certain electrode pins or the user may have dirt or grease on his scalp that affects the skin-electrode connections. In addition, the exact location of the electrodes on the user's head may vary from time to time. The ability of the selector 98 to utilize the most effective electrode points 40 within a large array overcomes both of these shortcomings.

Many variations of the invention within the scope of the appended claims will be apparent to those skilled in the art once the principles described herein have been read and understood.

What is claimed is:

1. A non-invasive skin surface electrode apparatus for placement adjacent to a mammalian body surface, said apparatus comprising,
a plurality of spaced apart projecting electrodes mounted upon a supporting base,
each being located on said supporting base toward so as to be able to engage a skin surface while in use,
each electrode has an electrically conductive surface positioned to be able to establish electrical contact with the skin during use,
the electrodes being mounted upon said supporting base in at least one cluster or in an array that places the projecting conductive surface of the electrodes in a flat or curved coplanar arrangement such that at least one the electrodes of each cluster or array is adapted to transmit electric current to the body of a mammalian or to contact an area of the skin surface thereof where electrical impulses arising from nerve or muscle contractions can be received by the electrodes, and
an electrical conductor wire connected to each electrode for conducting electric current to or from the body.

2. The skin surface electrode apparatus of claim 1 wherein the electrodes each have an elongated shaft and the electrode shafts are arranged in parallel relationship.

3. The apparatus of claim 1 wherein the electrodes are arranged in at least two spaced apart clusters for receiving electrical impulses from different parts of the body of the user to establish a signal differential between the two clusters.

4. The apparatus of claim 1 wherein the electrodes are arranged in an array adapted to cover an area larger than a body area where an electrical signal is likely to arise electrical transmission means connected between the wires and
a selector for identifying signals for use from particular electrodes with the array.

5. The apparatus of claim 4 wherein the selector provides a selection of electrodes based on predetermined signal characteristics and cuts out signals from the electrodes that have not been selected.

6. The apparatus of claim 5 wherein the selector circuit is constructed and arranged to select at least a pair of electrode clusters and
wherein each cluster produces signals having predetermined characteristics.

7. The apparatus of claim 1 wherein there are a pair of electrode clusters that produce signals that have a signal differential therebetween.

8. The apparatus of claim 1 wherein the sensor electrodes are arranged in an array of sufficient size to encompass at least one signal that arises from nerve impulses or muscle contractions within the body of the user and
each of the electrodes is connected to an electrical circuit for identifying electrodes that produce the largest signal-to-noise ratio for selecting a signal-to-noise ratio above a predetermined threshold and cutting off the signals from the remaining electrodes.

9. The apparatus of claim 8 wherein the selector is constructed and arranged to identify for use at least two clusters of different electrodes with a signal differential between the clusters and wherein both differential signals are above a predetermined signal-to-noise threshold.

10. The apparatus of claim 1 wherein the electrodes are mounted upon a headset with electrodes located on each side of the head,
the electrodes are arranged in a pair of vertically-spaced clusters that are positioned to contact the skin over peri-auricular muscles located generally above the ear and a second pair of clusters arranged horizontally that are positioned to contact the skin of the user over peri-auricular muscles located generally behind the ear of the user.

11. The apparatus of claim 10 wherein the headset includes a headband that extends over the top of the head.

12. The apparatus of claim 10 wherein the headset comprises an eyeglasses style headset including a portion adapted to contact the top of each ear, a bow portion that extend forwardly from the ear toward the front of the head during use and a forehead bar connecting a forward end of the bow portions with a nose contact support thereon.

13. The apparatus of claim 1 including a headset having a pair of laterally spaced apart generally upright electrode supporting base members positioned to be located on opposite sides of the head and
each base member including centrally directed sensor electrodes which are in an array of sufficient size to encompass peri-auricular muscles that are located both above and behind the ear on each side of the head and the electrodes are connected to an electronic selector for identifying signals from particular electrodes.

14. The apparatus of claim 13 wherein the selector includes circuitry that is constructed and arranged to identify signals from at least one cluster of electrodes based upon a selected signal-to-noise level that is above a predetermined threshold.

15. The apparatus of claim 1 wherein at least some of the electrodes are slidably mounted within an electrode assembly and are yieldably biased toward the user during use by means of a spring that is connected between the assembly and the at least some of the electrodes.

16. A non-invasive method of establishing electrical contact for signals traveling to or from a mammalian body skin surface comprising:
providing a plurality of spaced-apart skin contacting electrodes that together comprise an array of sufficient size to make contact with the surface of the body over a target area that contains at least one zone therein where electrical signals are to be applied or are capable of arising.
transmitting electrical signals to or from all of the electrodes in the array that are capable of establishing an electrical contact with the body through the skin,
sensing the quality of the electrical transmission in the electrodes throughout the array,
selecting electrodes in the array that establish an electrical connection of a quality above a selected threshold for continued transmission, and
cutting off the electrical connections to the electrodes that fall below the selected threshold,
such that it is unnecessary to precisely position the electrodes each time they are used by the same person or used by different persons.

17. The method of claim 16 wherein there are at least two zones within the array and,
wherein a signal differential exists between the two zones within the array.

18. The method of claim 16 including the step of providing as the selector a circuit for establishing the signal-to-noise ratio for each of the electrodes,
wherein an operational amplifier is wired for utilizing signals from the electrodes that are above the predetermined threshold while cutting off signals from the remaining electrodes.

19. The method of claim 16 including the step of:
providing a signal clipping or signal digitizing circuit.

20. The method according to claim 16 including the steps of:
supporting each electrode array for being located on either side of the head of the user to cover a region generally proximate the ear for receiving signals derived from peri-auricular nerves or muscles in at least a pair of zones having a signal differential between the pair of zones within the array,
selecting signals from the electrodes that are positioned over the zones where the peri-auricular signals arise and eliminating signals from the remaining electrodes,
whereby different selected electrodes are utilized to carry signals to an output device depending upon the position taken by the array of electrodes on the body of the user.

21. The method according to claim 16 wherein the electrodes are yieldably biased into contact with the skin surface between hairs.

22. The method according to claim 16 including the step of:
sensing the signal-to-noise ratio of electrical signals from the electrodes and
passing each signal through a selector that identifies each signal that has a signal-to-noise ratio that is above a predetermined threshold to thereby establish an electrode point or cluster of the best or most representative electrode signals and
transmitting signals thus selected from the electrode cluster or point to an output device.

23. The method of claim 22 wherein the signals are averaged before transmission to the output device.

24. The method of claim 22 wherein the signals are transmitted through a rule-based software-driven circuitry to select preferred signals for transmission to the output device.
